(19) United States
(12) Patent Application Publication
(10) Pub. No.: US 2021/0374981 A1
(43) Pub. Date: Dec. 2, 2021

(54) APPARATUS AND METHOD FOR WIDE-FIELD HYPERSPECTRAL IMAGING

(71) Applicant: Cancer Research Technology Limited, London (GB)

(72) Inventors: Jonghee YOON, London (GB); Sarah BOHNDIEK, London (GB)

(21) Appl. No.: 17/286,600

(22) PCT Filed: Oct. 16, 2019
(86) PCT No.: PCT/GB2019/052953

§ 371 Date: Apr. 19, 2021

(30) Foreign Application Priority Data
Oct. 19, 2018 (GB) 1817092.8

(51) Int. Cl. G06T 7/33; G01J 3/28; A61B 5/00; G06T 7/00

(52) U.S. Cl. CPC G06T 7/344; G01J 3/2823; A61B 5/0075; G06T 2207/30096; G06T 2207/10068; G06T 2207/30024; G06T 7/0012

(57) ABSTRACT

Embodiments of the present invention provide a hyperspectral endoscope system, comprising a memory for storing data therein, an endoscope arranged to, in use, receive radiation reflected from a sample and to output wide-field image data and line-scan hyperspectral data corresponding to the sample, a processor coupled to the memory, wherein the processor is arranged, in use, to determine registration information between portions of the wide-field image data, and determine wide-area hyperspectral image data in dependence on the registration information and the line-scan hyperspectral data.

### BACKGROUND

Hyperspectral imaging is used in various applications including investigating diseases in subjects. However, it is difficult to generate hyperspectral images in some applications, such as in endoscopy, because of a need for rapid data acquisition and the presence of image distortions. In endoscopy, the image distortions may arise from freehand imaging i.e. human control of an endoscope producing image data.

It is an object of embodiments of the invention to at least mitigate one or more of the problems of the prior art.

### BRIEF DESCRIPTION OF THE DRAWINGS

Embodiments of the invention will now be described by way of example only, with reference to the accompanying figures, in which:

FIG. 1 shows a schematic illustration of an endoscope system according to an embodiment of the invention;

FIG. 2 shows an illustration of a processing system according to an embodiment of the invention;

FIG. 3 shows a method according to an embodiment of the invention;

FIG. 4 shows wide-field image data according to an embodiment of the invention;

FIG. 5 shows line-scan hyperspectral image data according to an embodiment of the invention;

FIG. 6 shows a method according to another embodiment of the invention;

FIG. 7 shows a method according to a still further embodiment of the invention;

FIG. 8 illustrates wide-field images having identified features according to an embodiment of the invention;

FIG. 9 illustrates co-registered wide-field images according to an embodiment of the invention;

FIG. 10 shows co-registered wide-field images according to an embodiment of the invention;

FIG. 11 illustrates formation of wide-area hyperspectral image data according to an embodiment of the invention;

FIG. 12 illustrates a method according to an embodiment of the invention;

FIG. 13 illustrates formation of wide-area hyperspectral image data according to an embodiment of the invention;

FIG. 14 illustrates wide-area hyperspectral imaging of a vascular tree phantom with an embodiment of the invention;

FIG. 15 illustrates wide-area hyperspectral imaging using an embodiment of the invention of ex vivo tissue from a patient illustrating different tissue types; and

FIG. 16 illustrates wide-area hyperspectral imaging under clinical-mimicking conditions using an embodiment of the invention of an intact pig oesophagus.

### DETAILED DESCRIPTION OF EMBODIMENTS OF THE INVENTION

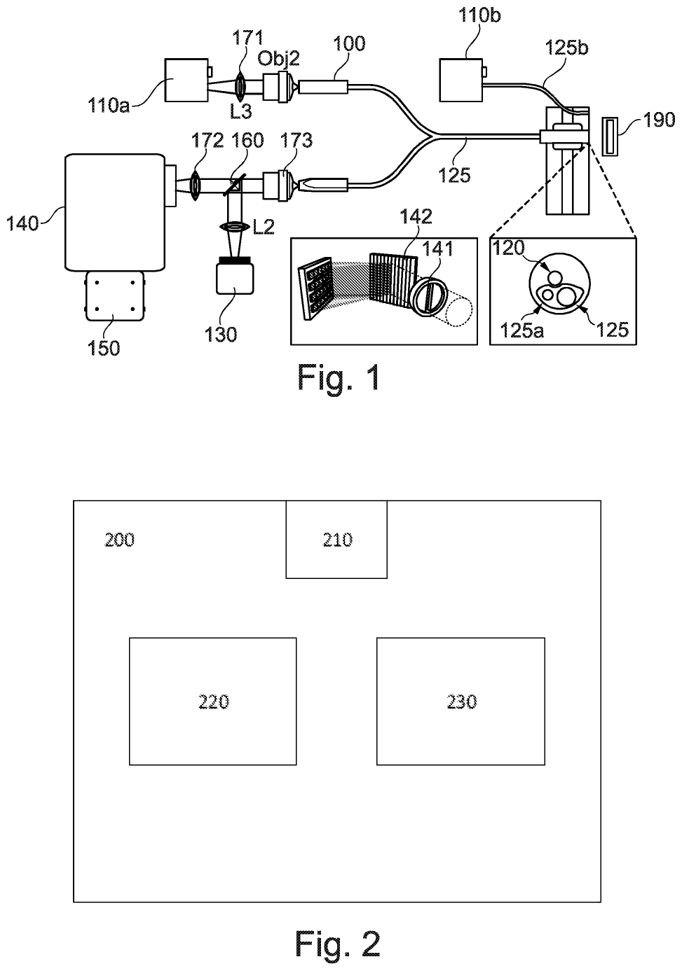

FIG. 1 illustrates a hyperspectral endoscope 100 according to an embodiment of the invention. The endoscope 100 is associated with a source of radiation 110a, 110b. The source of radiation 110a, 110b may either be comprised in the endoscope 100, as in the case of source 110a or may be a source 110b of radiation which is external to the endoscope 100 i.e. the source of radiation 110a, 110b is associated but not comprised within the endoscope 100. In either case, the source of radiation 110a, 110b may be a broadband source of radiation such as light i.e. white light.

The endoscope 100 comprises an imaging fibre 120 which is arranged to, in use, receive radiation reflected from a sample 190. In some embodiments, the imaging fibre 120 is an imaging fibre bundle 120 comprising a plurality of optical fibres for receiving radiation from the sample 190. In some embodiments the endoscope 100 comprises an illumination fibre 125a, 125b for communicating radiation from the source of radiation 110a, 110b toward the sample. The illumination fibre 125a may be associated with the imaging fibre 120 i.e. running adjacent thereto, such as in the case of the endoscope comprising the source of radiation 110a, or may be a separate illumination fibre 125b, particularly in the case of the source of radiation 110b being external to the endoscope 100. The imaging fibre bundle 120 and the illumination fibre 125a may be formed within a flexible body 125 of the endoscope 100.

The endoscope 100 further comprises an imaging device 130 for outputting wide-field image data, a spectrograph 140 for determining a spectrum of the radiation reflected from the sample 190. A further imaging device 150 may be associated with the spectrograph for outputting line-scan hyperspectral data. The imaging device 130 for outputting the wide-field image data may be a first imaging device 130 and the imaging device 150 associated with the spectrograph 140 may be referred to as a second imaging device 150. One or both of the first and second imaging devices 130, 150 may be CCDs or the like. The first imaging device may be a monochrome or colour imaging device.

The first imaging device 130 is utilised for determining registration information between frames of the wide-field image data as will be explained. In some embodiments the registration information comprises one or a plurality of transforms associated with respective portions of the wide-field image data. The wide-field image data comprises data in two axes, i.e. x and y axes, representative of the sample 190. During imaging using the endoscope 100, an end of the imaging fibre 120 is moved with respect to the sample 190. Thus frames of the wide-field image data represent the sample 190 at different locations of the imaging fibre 120. The movement of the imaging fibre 120 with respect to the sample 190 may comprise one or more of translation, rotation and magnification, as will be explained. Thus the one or more transforms may represent one or more of the translation, rotation and magnification as will be appreciated,

The spectrograph 140 may comprise an entrance slit 141 for forming a slit of incident radiation and a wavelength-separating device 142 for separating the slit of radiation in dependence on wavelength. The wavelength-separating device 142 may be a diffraction grating 142. Radiation separated according to wavelength is directed onto the second imaging device 150 which outputs line-scan hyperspectral data. The line-scan hyperspectral data comprises data in a first axis, such as ay-axis, representing the sample 190 and data in a second axis, such as the x-axis, representing wavelength.

The endoscope 100 comprises a beamsplitter 160 which splits or divides received radiation communicated along the imaging fibre 120 from the sample with a first portion of the radiation being directed to the first imaging device 130 for producing the wide-field image data and a second portion of the radiation being directed to the spectrograph 140 and the second imaging device 150 for producing the hyperspectral data. Thus the wide-field image data and hyperspectral data share distortion caused by the imaging system of the endoscope 100, which advantageously enables the wide-area hyperspectral data to be determined to account for said distortion. The endoscope 100 may further comprise one or more lenses for 171, 172, 173 for focusing incident radiation as will be appreciated.

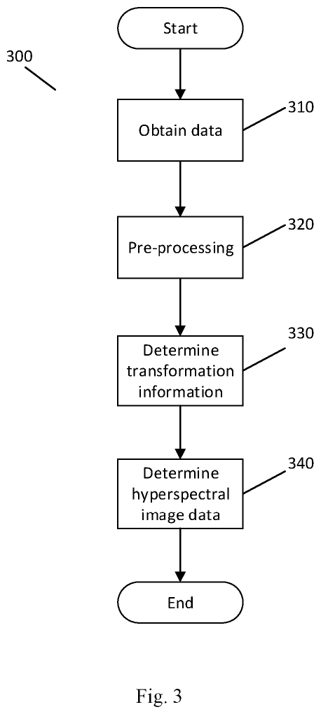

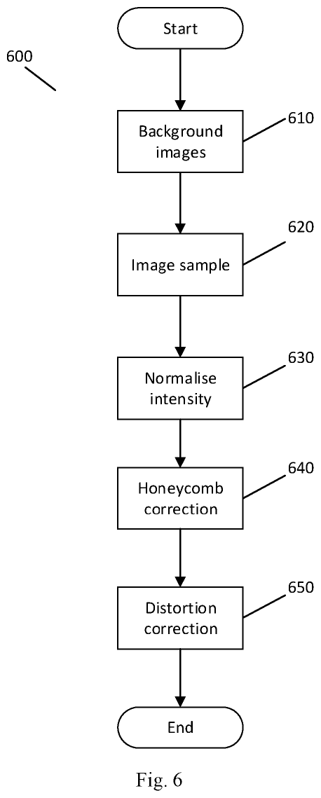

FIG. 2 illustrates a processing system 200 according to an embodiment of the invention. Data output by the endoscope 100 during use is provided to the processing system 200. The processing system 200 comprises an interface 210 for receiving data from the endoscope 100. The interface 210 may be a wired or wireless interface 210 for receiving data from the endoscope 100. The data comprises the wide-field image data and the line-scan hyperspectral data. The processing system 200 further comprises a memory 220 for storing the received data therein, which may be formed by one or more memory devices 220, and a processor 230 for processing the stored data by a method according to an embodiment of the present invention such as illustrated in FIGS. 3-12. The processor 230 may be formed by one or more electronic processing devices which operatively execute computer-readable instructions which may be stored in the memory 220.

FIG. 3 illustrates a method 300 according to an embodiment of the invention. The method 300 is a method of determining wide-area hyperspectral image data according to an embodiment of the invention. As described above, the endoscope 100 provides to the processing system 200 wide-field image data representative of the sample in two spatial axes, whereas the line-scan hyperspectral data is representative of wavelength against one of the two spatial axes i.e. the wide-field and line-scan hyperspectral data only share one common spatial axis, which may be the y-axis. The wide-area hyperspectral image data combines data in all of the three axes i.e. x, y and A. The combined data may be in the form of a 3D hypercube representing the wide-area hyperspectral image data.

The method 300 comprises a step 310 of obtaining data. In some embodiments the data comprises data relating to one or more references surfaces. The data relating to the one or more references surfaces may be obtained in a first portion of step 310. The obtained data comprises data relating to the sample 190 which is obtained using the endoscope 100, which may be obtained in a second portion of step 310. It will be understood that the first and second portions of data representing the one or more reference surfaces and the sample, respectively, may be obtained at different times.

In the first portion of step 310 the one or more reference surfaces are of predetermined brightness. Step 310 may comprise obtaining data relating to the one or more reference surfaces which are white and dark backgrounds for calibration. W_{white}, W_{dark}, S_{white}, and S_{dark} represent measurements of white and dark backgrounds wherein W is indicative of the data being wide-field image data and S is line-scan hyperspectral data. The white backgrounds may be measured by using a standard white reflectance target and light source, and the dark backgrounds may be measured with a camera shutter closed.

The second portion of step 310 comprises moving an end of the imaging fibre 120 with respect to the sample 190. The data comprises a plurality of frames of wide-field image data which may be referred to as W(i), wherein W is indicative of the data being wide-field image data and i is an index number of the data i.e. an index of the frame number where i=1, 2, 3 . . . n where n is a total number of frames of the data. The data comprises S(i) where S is line-scan hyperspectral data and i is the index number. As the endoscope is being moved with respect to the sample 190 during capture of the data, i represents an imaging position with respect to the sample 190. The wide-field image data and the line-scan hyperspectral data share a common or global spatial coordinate system.

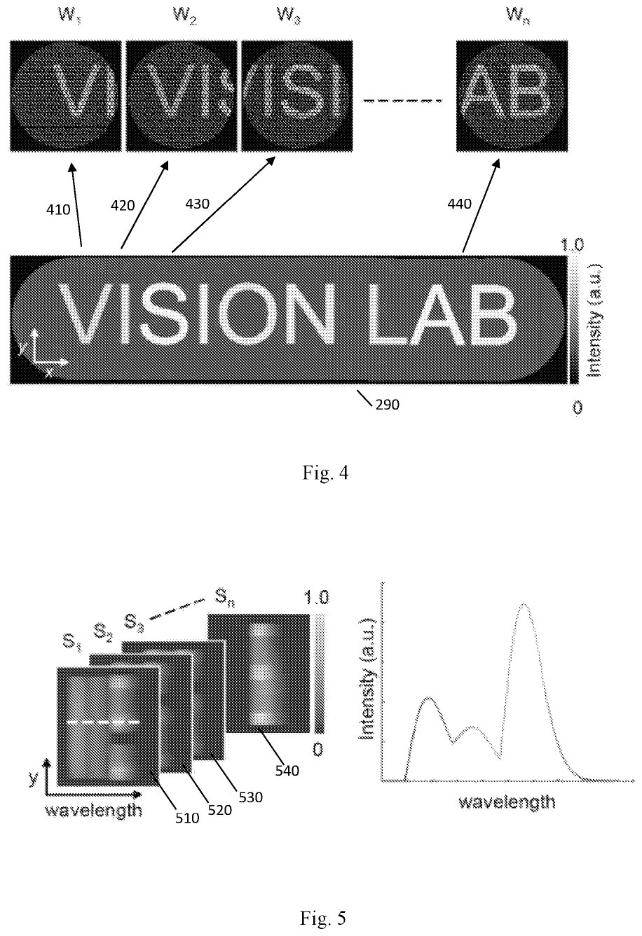

FIG. 4 illustrates a plurality of frames 410, 420, 430, 440 of the wide-field image data corresponding to sample 290. Illustrated in FIG. 4 are frames W₁, W₂, W₃, . . . Wₙ. Each frame 410, 420, 430, 440 of the wide-field image data is a two dimensional image frame 410, 420, 430, 440 providing image data in two axes of the sample i.e. x, y. As can be appreciated, each of the frames 410, 420, 430, 440 corresponds to a respective portion of the surface of the sample 290 from which reflected radiation is received. In the illustrated example the wide-field image data is monochrome comprising a value indicative of an intensity of radiation for each pixel, although it will be appreciated that colour image data may be used.

FIG. 5 illustrates line-scan hyperspectral data 510, 520, 530, 540 for a plurality of locations about the sample 290 illustrated in FIG. 4. The locations correspond to those of the frames 410, 420, 430, 440 shown in FIG. 4. Each line-scan hyperspectral data image 510, 520, 530, 540 is a two-dimensional image frame comprising data corresponding to one of the spatial axes of the wide-field image data 410, 420, 430, 440 i.e. corresponding to a surface of the sample 290, and an axis indicative of wavelength. Thus the line-scan hyperspectral data 510, 520, 530, 540 is representative of a wavelength disruption of an elongate portion of the sample 290 as imaged through the entrance slit 141.

Returning to FIG. 3, the method 300 comprises a step 320 of pre-processing the data obtained in step 310. The pre-processing may comprise one or more of intensity normalisation, structure or artefact removal and distortion removal.

The intensity normalisation may be performed in dependence on the data relating to the surface of predetermined brightness obtained in the first portion of step 310 in some embodiments. In step 320 intensity normalisation of the wide-field image data may be performed in dependence on the wide-field image data corresponding to surfaces of predetermined brightness. The intensity normalisation of wide-field image Wₓ may be performed to provide normalised wide-field image data NWₓ in some embodiments according to the following equation:

$$N {W}_{1} ( i ) = \frac{W ( i ) - {W}_{d a r k}}{{W}_{white} - {W}_{d a r k}} \qquad$$

Similarly, in some embodiments, intensity normalisation of the line-scan hyperspectral image data may be performed in step 320. In step 320 the intensity normalisation of the line-scan hyperspectral image data may be performed in dependence on the line-scan hyperspectral image data corresponding to the surfaces of predetermined brightness. The intensity normalisation of the line-scan hyperspectral image data, S(i) may be performed in some embodiments according to the following equation:

$$N S ( i ) = \frac{S ( i ) - {S}_{d a r k}}{{S}_{white} - {S}_{d a r k}} \qquad$$

Which produces an intensity normalised line-scan hyperspectral image NS(i). NW₁(i) may be used to denote a wide-field image and NS(i) a line-scan hyperspectral image after intensity normalisation.

In some embodiments, step 320 comprises removing honeycomb structures from the wide-field image data. The honeycomb structures may be removed by applying low-pass filtering to the normalised wide-field image data. The honeycomb structures may be removed using low-pass Fourier filtering of NW₁(i), which removes high frequency components from the image data including peaks arising due to the structure of the imaging fibre 120. A cut-off frequency of the low-pass filter used may be determined in dependence on image sizes and multicore imaging fibre bundle structures. The low-pass filtering may be performed in Fourier space (frequency domain) by removing information out of a low-pass filtering mask. Thus, scales in Fourier space may be determined in dependence upon an original size of the wide-field image data. In some embodiments a size of the low-pass filtering mask may be determined based on a size of the endoscopic image and imaging fibre core. NW₂(i) may be used to denote a wide-field image after removal of honeycomb structures.

In some embodiments, step 320 comprises correcting for, or reducing, barrel distortion which may be caused by varying degrees of magnification along a radial axis. The barrel distortion may be corrected for according to the equation:

x_{c}=x₀+αr cos θ

y_{c}=y₀+αr sin θ

where x_{c} and y_{c} are corrected locations or pixels of NW₃(i), x₀ and y₀ are a centre position of image NW₂(i), r is a radial distance from (x₀, y₀) to (x,y) in polar coordinate, θ is an angle between x-axis and line from (x₀,y₀) to (x,y), and a is a correcting coefficient. NW₃(i) is wide-field image data after correcting for the barrel distortion.

The method 300 comprises a step 330 of determining the registration information. The registration information is determined in step 330 in dependence on the wide-field image data. The registration information is indicative of one or both of an imaging position and a distortion of each wide-field image frame 410, 420, 430, 440. In some embodiments the registration information is the one or more transforms which each may be a geometric transform matrix. In some embodiments a transform is associated with each respective wide-field image. Thus the method 300 may comprise determining a plurality of geometric transformation matrices (GMs) between wide-field images 410, 420, 430, 440 as will be explained. Each GM has predetermined dimensions which, in an example embodiment, are 3×3, although it will be appreciated that GMs having other dimensions may be used.

A GM represents a transformation matrix, such as a 3×3 matrix, which includes 2D transformation information of scale, shear, rotation, and translation. A GM may be defined in Projective, Affine, Similarity, and Euclidian spaces. Transformation of an image using a GM may be performed using the following equation:

$$[ \begin{matrix}{x}^{′} \\ {y}^{′} \\ 1\end{matrix} ] = [ \begin{matrix}t 11 & t 12 & t 13 \\ t 21 & t 22 & t 23 \\ t 31 & t 32 & t 33\end{matrix} ] \times [ \begin{matrix}x \\ y \\ 1\end{matrix} ]$$

where (x,y) and (x′,y′) represent spatial coordinates of original and corresponding points, i.e. pixels, in the transformed image, respectively, and the 3×3 matrix represents the GM. A 4×4 GMs may be used in 3D Projective and Affine spaces as desired.

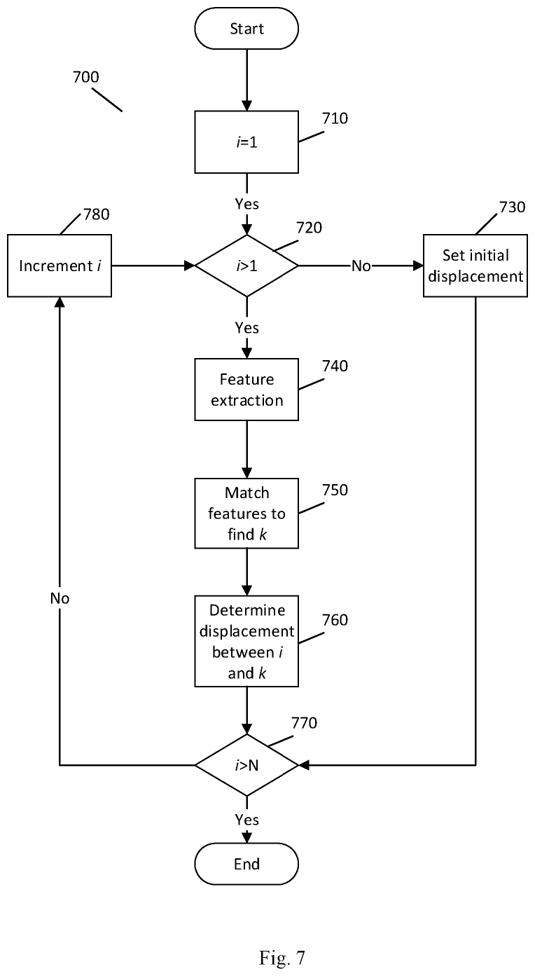

FIG. 7 illustrates a method 700 of determining the registration information according to an embodiment of the invention. The method 700 is operable on the wide-field image data 410, 420, 430, 440 to determine the registration information which, as noted above, may be one or more transforms.

In step 710 a reference wide-field image frame is selected. In the illustrated example the first wide-field image at location 1=1 is selected as the reference image. In step 720 it is considered whether the value of i is greater than 1 which, in the first iteration of step 720 in the example, is negative given that i is set to 1 in step 710. Thus the method moves to step 730 where an initial displacement is determined. The initial displacement is set in step 730 based on the reference wide-field image i.e. 1=1.

Step 730 may comprise, in some embodiments, setting an initial GM to one or more predetermined values. Each GM may be referenced as GM(i) corresponding to one of the wide-field images with which it is associated. Thus in step 730 GM(i) which in step 730 is GM(1) may be set to predetermined values, which may be:

$$GM ( 1 ) = [ \begin{matrix}1 & 0 & 0 \\ 0 & 1 & 0 \\ 0 & 0 & 1\end{matrix} ] \qquad$$

The initial GM i.e. GM(1) is used in embodiments of the invention to determine relative registration information i.e. other transforms or GMs relative to GM(1).

Following step 730 the method moves to step 770 where it is determined whether i is less than a predetermined value n. The predetermined value is indicative of a total number of wide-field images i.e. n is the total number of frames of the data as explained above. If i is less than n, the method moves to step 780 where the value of i is incremented i.e. in the example for the first iteration of step 780 i is incremented to a value of i=2. Thus i is used to select a next wide-field image which in the example embodiment is a next successive i.e. 2^{nd} wide-field image. In a second iteration of step 720 i is greater than 1 and thus the method moves to step 740.

In step 740 a feature extraction algorithm is utilised to identify features in each of the wide field images. In some embodiments a Speeded Up Robust Features (SURF) algorithm may be used in step 740, although in other embodiments other feature extraction algorithms, such as a Scale-invariant feature transform or a Maximally stable extremal regions algorithm, may be used. It will be appreciated that other feature extraction algorithms may be used.

Step 750 comprises determining one of the wide-field images k having one or more matching features to the currently selected wide-field image i.e. NW₃(i). Thus, as result of steps 740 and 750, the feature extraction algorithm is used in some embodiments to find a best matching wide-field image NW₃(k) to NW₃(i). The best matching may be having a most number of common features between the wide-field images.

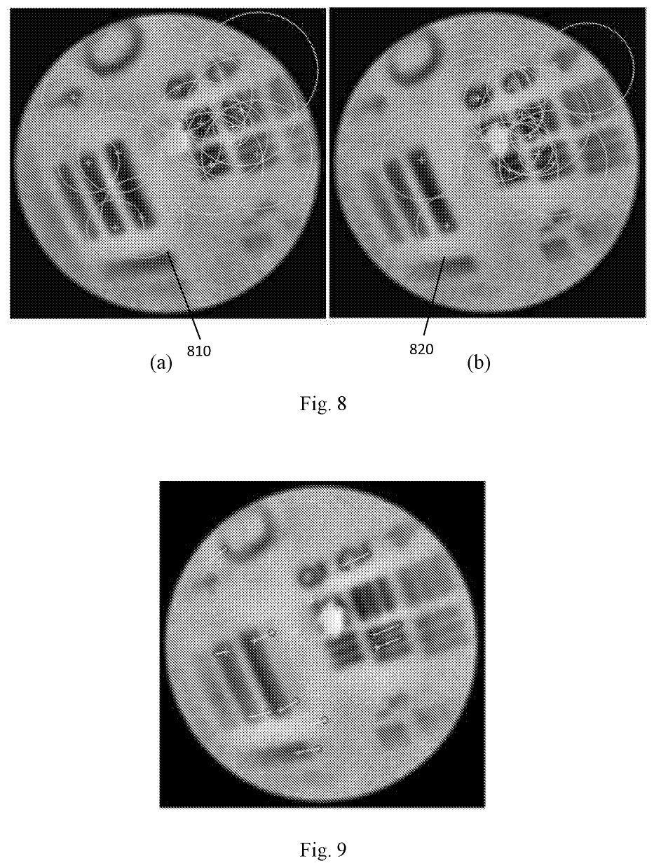

FIG. 8(a) illustrates a current image NW₃(i) and FIG. 8(b) is another wide-field image NW₃(k) both having had a feature extraction algorithm applied to identify features present in each image. Features in each image are indicated with rings. A first feature is identified in FIG. 8(a) as 810 and the same feature is identified in FIG. 8(b) as 820. It will be appreciated that FIGS. 8a and 8b are from different imaging positions, thus the location of feature 810, 820 is moved between FIGS. 8a and 8b, as illustrated in FIG. 9 which shows an association of, and translation of, features between the images NW₃(i) and NW₃(k).

In step 760 a transform is determined between the wide field images determined in step 750. That is, a transform is determined in step 760 between the wide-field images NW₃(i) and NW₃(k). In some embodiments, step 760 comprises determining a relative GM between the images NW₃(i) and NW₃(k). The relative GMᵣ is associated with the wide field image i, GMᵣ(i) may be determined by optimising global spatial coordinates. The global spatial coordinates are coordinates used over all wide-field images as a global set of coordinates.

Then a GM for the current wide-field image GM(i) may be determined as:

GM(i)=GMᵣ(i)×GM(k)

where GM(k) is a GM for the kth wide-field image.

For example, GM(i) may be determined as:

$$( \begin{matrix}1.11 & - 1.1 \times {10}^{- 4} & 0 \\ - 0.01 & 1.09 & 0 \\ 62.12 & 43.24 & 1\end{matrix} ) \times ( \begin{matrix}0.93 & 6.6 \times {10}^{- 3} & 0 \\ 2.6 \times {10}^{- 3} & 0.96 & 0 \\ - 11.84 & 15.65 & 1\end{matrix} ) = ( \begin{matrix}1.03 & 7.2 \times {10}^{- 7} & 0 \\ 2.94 \times {10}^{- 5} & 1.04 & 0 \\ - 737.52 & - 6 \; 76.71 & 1\end{matrix} ) \\ G M ( k ) \times {GM}_{r} ( i ) ( Relative GM between {NW}_{3} ( k ) and {NW}_{3} ( i ) ) = G M ( i ) \qquad$$

As GM(i) indicates a relative transformation between the current image NW₃(i) and NW₃(1) using the global spatial coordinates, a relative transform for all the wide-field images relative to NW₃(1).

The method then moves to step 770 as previously described. It will be appreciated that for each of the wide-field images 410, 420, 430, 440 registration information which may be the form of a respective transform such as a GM is determined by the method 700.

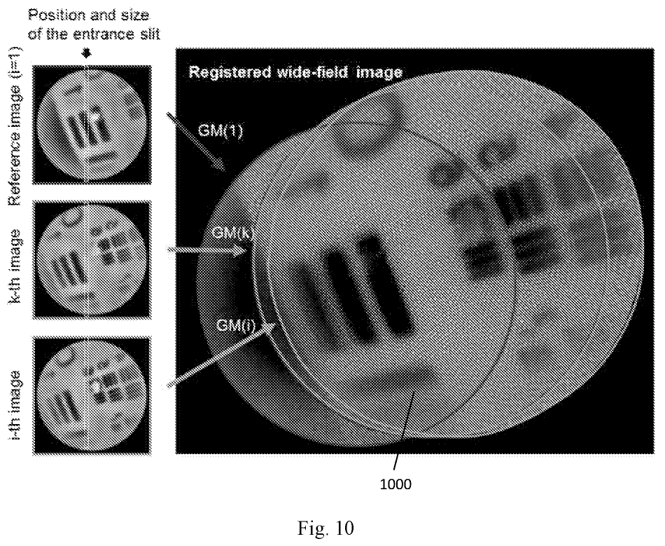

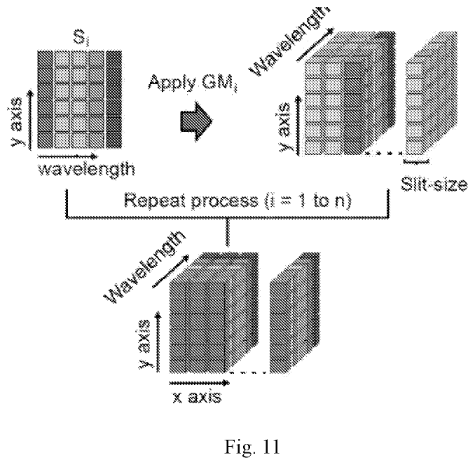

As a result of the method 700 a registered wide-field image 1000 may be produced representing a combination of a plurality of individual wide-field images 410, 420, 430, 440 where registration information determined by the method 700 is utilised to register the individual wide-field images 410, 420, 430, 440. Illustrated in FIG. 10 is three wide field images i=1, an i^{th} image and a k^{th} image. As can be appreciated, the individual wide-field images are combined with respective transformations to form the registered wide-field image 1000. FIG. 10 also shows a position and size of the entrance slit 141 relative to each wide-field image.

Returning again to FIG. 3, the method 300 comprises a step 340 of determining the wide-area hyperspectral image data. The wide-area hyperspectral image data comprises three dimensions corresponding to x, y and λ. In some embodiments the wide-area hyperspectral image data is a hypercube. The wide-area hyperspectral image data is determined in dependence on the line-scan hyperspectral image data and the registration information determined by an embodiment of the method 700. FIG. 11 illustrates a process of forming the hypercube from the line-scan hyperspectral image data according to an embodiment of the invention.

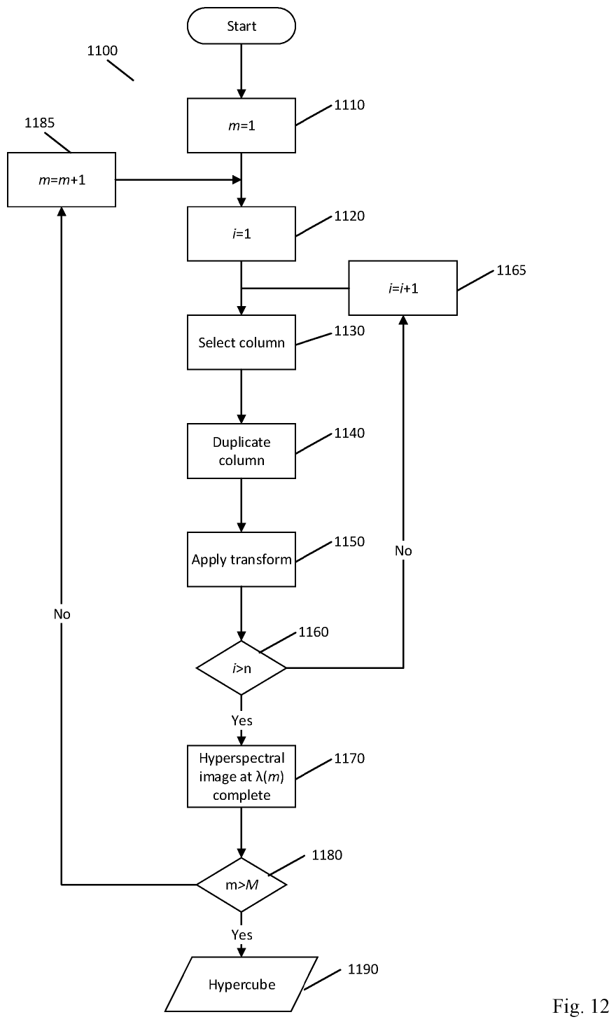

An embodiment of a method 1100 is illustrated in FIG. 12 which may be performed in step 340 of the method 300 shown in FIG. 3.

In step 1110 of the method 1100 a first wavelength of radiation represented in the line-scan hyperspectral image data is selected. The wavelength may be selected according to an index m as λ(m). In the example method 1100 m=1 in step 1110.

In step 1120 a first line-scan hyperspectral image i is selected. In the example, the first line-scan hyperspectral image i is i=1, although it will be appreciated that other images may be selected as the first image in step 1120.

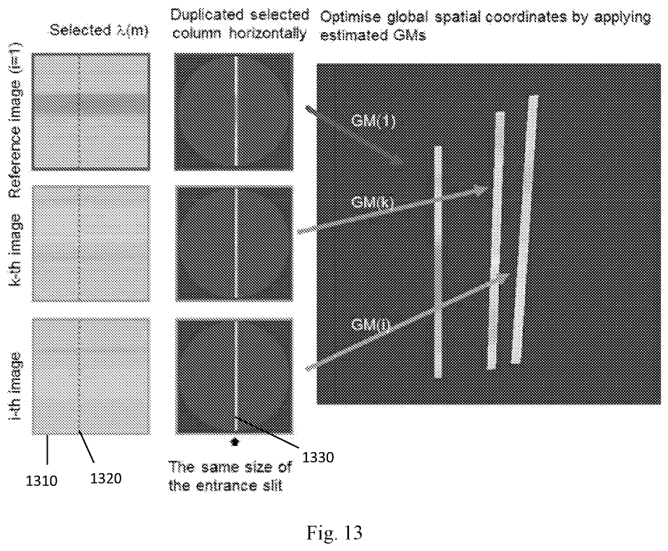

Referring to FIG. 13, reference line-scan hyperspectral images 1310 are shown in the left-hand column. Reference line-scan hyperspectral images 1, k and i are shown. A dotted vertical line 1320 on each image illustrates the wavelength m selected in step 1110.

In step 1130 a column of hyperspectral data of the currently selected line-scan hyperspectral image i is selected according to the currently selected wavelength m. That is, a column 1320 of the hyperspectral image data from the line-scan hyperspectral image NS(i) corresponding to λ(m) is selected in step 1130. The column 1320 of hyperspectral image data corresponds to that in the hyperspectral image NS(i) corresponding to the dotted line in FIG. 13. The column of hyperspectral image data has integrated spectral information along the x-axis due to the finite size of the slit 141 and grating 142 inside the spectrograph 140.

Step 1140 comprises duplicating the column of the hyperspectral image data selected in step 1130. The selected column of line-scan hyperspectral image data is one-dimensional, for example in they-axis corresponding to the selected wavelength m and is duplicated in step 1140 a second dimension. The duplication may be along the x-axis of the three-dimensional hyperspectral image data. The selected column of line-scan hyperspectral image data may be duplicated to match a physical size, i.e. width in the x-axis, of the entrance slit 141 and produce duplicated line-scan hyperspectral data DS(i) which is two dimensional i.e. in both x- and y-axes. Thus, the duplicated hyperspectral data matches dimensions of the entrance slit 141. A middle column of FIG. 13 illustrates duplicated hyperspectral image data 1330 matching the entrance slit 141 size.

In step 1150 a transform corresponding to that of the image i is applied to the duplicated hyperspectral image data to transform said data. For example, the duplicated hyperspectral image data is positioned on the global spatial coordinates according to the transform. In particular, step 1150 may comprise transforming a created 2D matrix DS(i) onto a set of global spatial coordinates by applying the estimated GM(i) associated with the image i using the equation discussed above:

$$[ \begin{matrix}{x}^{′} \\ {y}^{′} \\ 1\end{matrix} ] = [ \begin{matrix}t 11 & t 12 & t 13 \\ t 21 & t 22 & t 23 \\ t 31 & t 32 & t 33\end{matrix} ] \times [ \begin{matrix}x \\ y \\ 1\end{matrix} ]$$

In step 1160 it is determined whether i, corresponding to the currently selected image, is less than n representing the total number of frames of the data i.e. step 1160 determines whether all images have been considered. If not, i.e. i≤n then the method moves to step 1165 where a next image is selected which may comprise i being incremented before the method returns to step 1130 where a column of the hyperspectral image data corresponding to the wavelength m is selected. Thus steps 1130-1165 cause a column of the hyperspectral image data corresponding to the wavelength m to be selected from each of a plurality of hyperspectral images 1 . . . i . . . n. The selected columns are duplicated in some embodiments to match the entrance slit size and then transformed onto the global spatial coordinates to form an image for the wavelength m in x- and y-axes.

FIG. 13 illustrates the duplicated hyperspectral image data DS(1), DS(k) and DS(i) being transformed onto the global spatial coordinates by respective transforms GM(1), GM(k) and GM(i) as illustrated.

Once all n hyperspectral images have been considered in step 1160 the hyperspectral image at wavelength λ(m) is complete, as denoted by step 1170.

In step 1180 it is determined whether the currently selected wavelength m is the last wavelength i.e. m>M. If not i.e. there remain further wavelengths to be considered, the method moves to step 1185 where a next wavelength is selected. In some embodiments step 1185 comprises incrementing m i.e. to select the next wavelength. Following step 1185 the method moves to step 1120 where a first image is again selected for performing the remaining steps at the newly selected wavelength i.e. m+1.

If, however, at step 1180 the wavelength m was the last wavelength to be considered i.e. a maximum wavelength for constructing the hypercube, the wide-area hyperspectral image data is complete. In some embodiments, where the wide-area hyperspectral image data is a hypercube then the hypercube is complete as denoted by 1190 in FIG. 12.

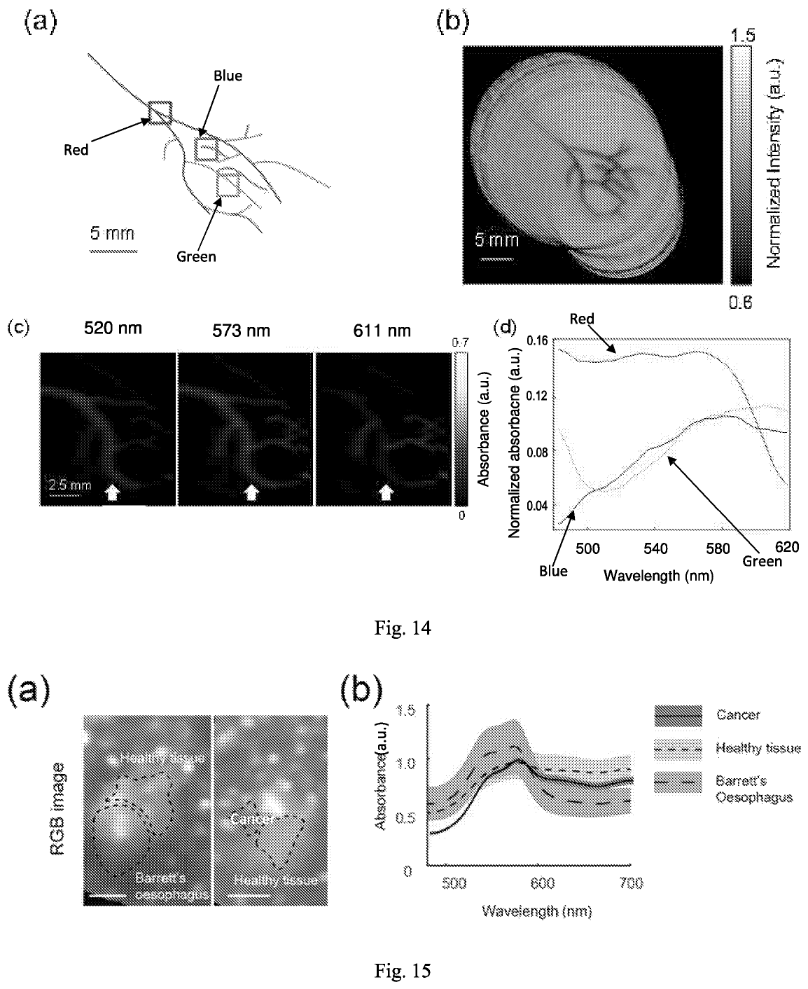

FIG. 14 illustrates in FIG. 14(a) a vascular tree phantom which was created using three colours to demonstrate free-hand hyperspectral endoscopic imaging with an embodiment of the invention. As shown in FIG. 14(b), during free-hand imaging of the phantom using an embodiment of the invention, wide-field registration was performed and a combination of 59 endoscopic wide-field images is shown. FIG. 14(c) shows representative slice images at three wavelengths from the reconstructed wide-area hyperspectral image data which is in the form of a hypercube. White arrows in FIG. 14(c) indicate the presence and absence of red vascular structures in the different single wavelength images. The colour bar at the right hand side of FIG. 14(c) indicates absorbance (a.u.). In FIG. 14(d) absorbance was quantified within the red, green, and blue squares shown in FIG. 14(a).

The images shown in FIG. 14 demonstrate embodiments of the invention enabling real-time hyperspectral imaging acquisition with free-hand operation of an endoscope according to an embodiment of the image. A colour vascular phantom was printed and measured by hyperspectral endoscopy with an acquisition time of 15 ms, which enables video-rate (26.2±0.2 fps) hyperspectral imaging. Registered wide-field images and representative slice images from the reconstructed hypercube as shown in FIGS. 14(b) & (c) demonstrate that embodiments of the present invention work well under video-rate hyperspectral imaging with free-hand motion. Moreover, spectral analysis shows that embodiments of the invention are able to measure a spectral profile of the sample accurately. Therefore, embodiments of the invention may be used to measure both spatial and spectral data rapidly and accurately during freehand motion. Advantageously this may be superior to known techniques such as multispectral imaging, snapshot imaging, and line-scan hyperspectral imaging with mechanical scanning units, such as a Galvano mirror.

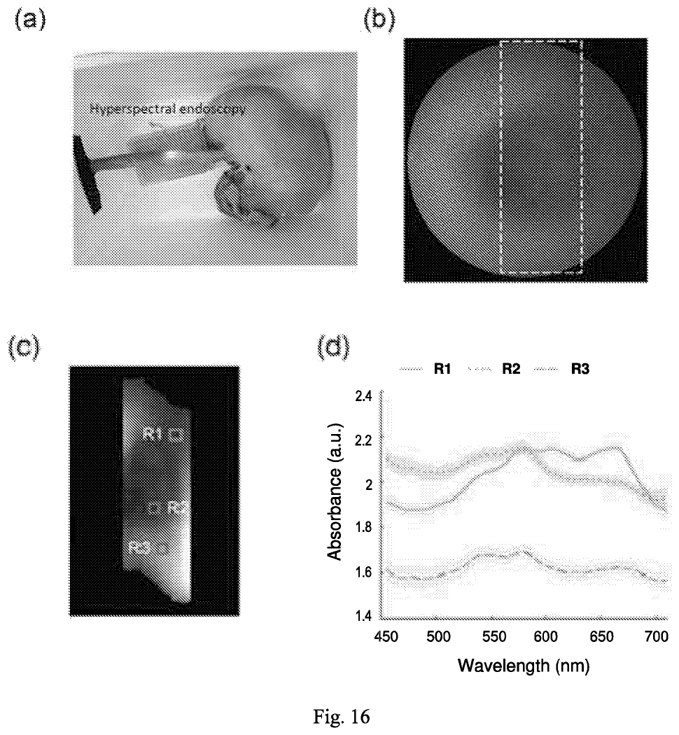

FIG. 15 shows hyperspectral imaging of ex vivo sample tissue from a patient, which may be a human patient. FIG. 15 shows a distinct spectral profile depending on tissue types which enables identification of the different tissue types and particularly, although not exclusively, cancerous tissue. FIG. 15(a) shows representative RGB images of two tissue samples. In each image a dashed line indicates a boundary of healthy tissue, Barrett's oesophagus, and cancer tissue, respectively. FIG. 15(b) indicates a spectrum of the identified tissue types shown in (a). Solid lines and shaded areas in (b) indicate mean value and standard deviation of the absorbance profile, respectively. Scale bars=1 mm.

FIG. 15 shows that embodiments of the invention have potential in clinical applications by measuring pathologic human tissues collected from the patients: healthy tissue, Barrett's oesophagus, and oesophageal cancer. The boundaries of each tissue type (dashed lines in RGB images) were selected based on histopathology analysis and the operating endoscopists. The average and standard deviation of absorption spectra in the selected areas of healthy tissue, Barrett's oesophagus, oesophageal cancer were extracted. As can be appreciated, there are distinct absorption spectra depending on the tissue type, which indicates that embodiments of the invention enable discrimination of healthy and diseased tissues, such as cancerous tissue, based on the spectral profile of the tissue or regions thereof. Therefore, embodiments of the present invention may comprise comparing the wide-area hyperspectral image data with one or more wavelength thresholds to determine the presence of cancer in the sample.

FIG. 16 illustrates wide-area hyperspectral imaging according to an embodiment of the invention under clinical-mimicking conditions of an intact pig oesophagus. FIG. 16(a) shows an experimental setup or apparatus according to an embodiment of the invention. The apparatus was introduced through the pig oesophagus to perform wide-area hyperspectral imaging of the pig oesophagus. The pig oesophagus was stained with a blue colour dye. The blue colour dye may be methylene blue. FIG. 16(b) shows a representative RGB image of the pig oesophagus. In FIG. 16(b), a dashed line indicates an area where hyperspectral imaging was performed using an embodiment of the invention. FIG. 16(c) illustrates a reconstructed wide-area hyperspectral image of the pig oesophagus measured from the area shown in (b). In addition, FIG. 16(c) illustrates three regions R1, R2, R3. Region R1 was stained with the blue colour dye and regions R2 and R3 were unstained. FIG. 16(d) indicates a spectrum of the three regions shown in (c). As shown in FIG. 15(d), R1 clearly shows a distinct spectral profile compared to regions R2 and R3. FIG. 15(d) illustrates a line and a shaded area for each region. The line and the shaded area for each region represent a mean value and a standard deviation of an absorbance profile indicated by the spectrum, respectively.

FIG. 16 demonstrates that embodiments of the invention have potential in clinical applications by measuring or producing wide-area hyperspectral imaging under clinical-mimicking conditions. The pig oesophagus was stained with the blue colour dye to demonstrate that embodiments of the present invention enable discrimination of tissue based on the spectrum. The mean value and the standard deviation of absorption spectra in the selected areas of the unstained and stained oesophagus were extracted. As can be appreciated, there are distinct absorption spectra depending on the blue colour dye staining, which indicates that embodiments of the invention enable discrimination of tissues based on the spectral profile. Therefore, embodiments of the present invention may discriminate healthy and diseased tissues based on the spectral profile under clinical conditions.

Embodiments of the invention therefore comprise a method of imaging tissue wherein wide-area hyperspectral image data corresponding to at least a portion of a tissue sample is produced using the system of an embodiment of the invention or using an embodiment of the invention as described above. The tissue sample may be imaged in vivo or ex vivo. Therefore, the tissue sample may be an in vivo tissue sample or an ex vivo tissue sample. Furthermore, the method may be an in vivo or an in vitro method of imaging tissue.

Embodiments of the invention furthermore comprise a method of diagnosing cancer in a subject, the method comprising producing wide-area hyperspectral image data corresponding to at least a portion of a tissue of the subject using the system of an embodiment of the invention or using a method according to an embodiment of the invention as described above.

The method may be a method performed in vivo. The tissue may be a tissue sample from the subject. Alternatively, the method may be performed in vitro. In such an embodiment, the sample may be an ex vivo tissue sample. Therefore, the method may be an in vitro method of diagnosing cancer.

The method comprises, in some embodiments, determining, in dependence on the wide-area hyperspectral image data, a presence of cancer in the tissue according to a wavelength of at least a portion of the image data. The method may comprise comparing the wide-area hyperspectral image data with one or more wavelength thresholds to determine the presence of cancer in the tissue.

In such embodiments, the method may further comprise providing treatment to the subject. The treatment may comprise cancer treatment. The cancer treatment may comprise a therapeutic agent for the treatment of cancer, suitably oesophagus cancer. The treatment may comprise administering a therapeutic agent to the subject. Suitable therapeutic agents may include: cisplatin, fluorouracil, capecitabine, epirubicin, oxaliplatin, irinotecan, paclitaxel, carboplatin, and the like.

Accordingly, the invention may comprise a method of treatment of cancer in a subject in need thereof, the method comprising:
(a) producing wide-area hyperspectral image data corresponding to at least a portion of a tissue of the subject using the system of an embodiment of the invention or using a method according to an embodiment of the invention as described above;
(b) determining, in dependence on the wide-area hyperspectral image data, a presence of cancer in the sample according to a wavelength of at least a portion of the image data;
(c) providing treatment to the subject with cancer.

A suitable sample for use in the methods of the invention is a tissue sample, suitably the tissue sample is derived from a biopsy of the relevant tissue, suitably the tissue sample is derived from a biopsy of the subject. The biopsy may be a biopsy from the oesophagus of the subject. Suitably therefore the tissue or tissue sample may be oesophagus tissue. In some embodiments, the methods of the invention may comprise obtaining a sample from a subject. Methods for obtaining such samples are well known to a person of ordinary skill in the art, such as biopsies.

The subject may be suspected of having cancer. Suitably the subject may be suspected of having oesophagus cancer. The subject may have or demonstrate symptoms of cancer. The subject may exhibit risk factors associated with oesophagus cancer.

The subject is suitably human.

The methods of the invention may be for diagnosing or treatment of cancers of the gastro-intestinal tract, suitably for diagnosing or treatment of oesophagus cancer.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine readable storage storing such a program. Still further, embodiments of the present invention may be conveyed electronically via any medium such as a communication signal carried over a wired or wireless connection and embodiments suitably encompass the same. The medium may be tangible or non-transitory.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

1. A hyperspectral endoscope system, comprising:
a memory for storing data therein,
an endoscope arranged to, in use, receive radiation reflected from a sample and to output wide-field image data and line-scan hyperspectral data corresponding to the sample;
a processor coupled to the memory, wherein the processor is arranged, in use, to:
determine registration information between portions of the wide-field image data; and
determine wide-area hyperspectral image data in dependence on the registration information and the line-scan hyperspectral data.

2. The system of claim 1, wherein the registration information is determined with respect to first and second portions of wide-field image data at respective first and second locations of the endoscope with respect to the sample.

3. The system of claim 2, comprising selecting the first and second portions of wide-field image data according to a predetermined feature matching algorithm.

4. The system of claim 1, wherein the registration information comprises transformation information of one or more of scale, shear, rotation, and translation.

5. The system of claim 1, wherein the determining the wide-area hyperspectral image data comprises selecting a portion of the line-scan hyperspectral image data corresponding to a portion of the wide-field image data associated with respective registration information.

6. The system of claim 1, wherein the determining the wide-area hyperspectral image data comprises selecting a portion of the line-scan hyperspectral data in dependence on wavelength.

7. The system of claim 6, comprising duplicating the selected portion of the line-scan hyperspectral data according to one or more predetermined conditions.

8. The system of claim 7, wherein the one or more predetermined conditions comprise matching one or more dimensions of the selected portion of the line-scan hyperspectral image data to an entrance slit of the endoscope.

9. The system of claim 6, comprising registering the selected portion of the line-scan hyperspectral data in dependence on the registration information.

10. The system of claim 9, wherein the registering the selected portion of the line-scan hyperspectral data comprises transforming the selected portion onto a global coordinate system according to a transform associated with a corresponding wide-field image.

11. The system of claim 6, wherein the portion of the line-scan hyperspectral data selected in dependence on wavelength is associated with a respective portion of the wide-field image data.

12. The system of claim 6, comprising selecting a plurality of portions of the image data in dependence on wavelength to form a wavelength dimension of a hypercube forming the wide-area hyperspectral image data.

13-14. (canceled)

15. A method of producing wide-area hyperspectral image data from an endoscope, comprising:
determining registration information between portions of wide-field image data received from an endoscope arranged to, in use, receive radiation reflected from a sample and to output the wide-field image data and line-scan hyperspectral data corresponding to the sample;
determining wide-area hyperspectral image data in dependence on the registration information and the line-scan hyperspectral data.

16. The method of claim 15, wherein registration information is determined with respect to the first and second portions of the wide-field image data at respective first and second locations of the endoscope with respect to a sample.

17. The method of claim 16, comprising selecting first and second portions of the wide-field image data according to a predetermined feature matching algorithm.

18. The method of claim 15, wherein registration information comprises transformation information of one or more of scale, shear, rotation, and translation.

19. The method of claim 15, wherein determining wide-area hyperspectral image data comprises selecting a portion of line-scan hyperspectral image data corresponding to a portion of wide-field image data associated with respective registration information.

20. The method of claim 15, wherein the determining wide-area hyperspectral image data comprises selecting a portion of the line-scan hyperspectral data in dependence on wavelength.

21-27. (canceled)

28. A method of diagnosing cancer in a subject, the method comprising:
producing wide-area hyperspectral image data corresponding to at least a portion of a tissue sample from the subject using the system of claim 1; and
determining, in dependence on the wide-area hyperspectral image data, a presence of cancer in the sample according to a wavelength of at least a portion of the image data.

29. The method of claim 28, comprising comparing the wide-area hyperspectral image data with one or more wavelength thresholds to determine the presence of cancer in the sample.

30. The method of claim 28, wherein the tissue sample is an ex vivo tissue sample.

31. (canceled)

32. A non-transitory computer readable medium having computer-executable instructions stored thereon which, when executed by a computer, is arranged to perform a method according to claim 15.
